(19) United States
(12) Patent Application Publication
(10) Pub. No.: US 2004/0220115 A1
(43) Pub. Date: Nov. 4, 2004

(54) Medicinal compositions and their method of preparation

(76) Inventor: Bill E. Cham, Sheldon (AU)

Correspondence Address: STITES & HARBISON PLLC, 1199 NORTH FAIRFAX STREET, SUITE 900, ALEXANDRIA, VA, 22314, US

(21) Appl. No.: 10/752,095

(22) Filed: Jan. 7, 2004

Related U.S. Application Data
(63) Continuation of application No. 09/958,333, filed on Dec. 20, 2001, now abandoned.
Application No. 09/958,333 is a 371 of international application No. PCT/AU00/00300, filed on Apr. 10, 2000.

(30) Foreign Application Priority Data
Apr. 9, 1999 (AU) PP 9686

(51) Int. Cl. A61K 31/7048; A61K 31/58

(52) U.S. Cl. 514/26; 514/176

(57) ABSTRACT

A medicinal composition comprising at least one compound which can interact with a target cell, the at least one compound being a glycoalkaloid of general formula (I) wherein: the composition is essentially without free sugars of the type which inhibit the interaction between the at least one glycoalkaloid and a target cell.

This application is a continuation of application Ser. No. 09/958,333, which is the National Stage of International Application No. PCT/AU00/00300, filed Apr. 10, 2000.

### FIELD OF THE INVENTION

The present invention relates to medicinal compositions and in particular therapeutic compositions comprising glycoalkaloids. Such compositions may be used in the treatment, control and diagnosis of cancers and tumors in mammals, contraception and termination of pregnancy. The present invention is particularly directed towards a composition comprising a mixture of solasodine glycosides.

The present invention is also directed towards a method of preparing a medicinal composition and a method of treatment, control or diagnosis of cancers and tumors in mammals.

### BACKGROUND ART

Glycoalkaloids are steroidal alkaloids which have a sugar moiety bound to the alkaloid moiety. The sugar moiety can be a monosaccharide, disaccharide, oligosaccharide or polysaccharide. Certain glycoalkaloids derived from plants have been observed to have anti-cancer properties.

Of particular interest are glycoalkaloids extracted from the Solanum genus. Glycoalkaloids from the species Solanum Sodomaeum L. have been shown to be active against cancer in animals and skin tumors in humans. The glycoalkaloids extracted from the fruit of Solanum Sodomaeum L. include the triglycosides solasonine,[(22R, 25R)-spiro-5-en-3β-yl-α-L-rhamnopyranosyl-(1->2gal)-O-β-D-glucopyranosyl-(1->3gal)-β-D-galactopyranose](33%), solamargine′(22R,25R)-spiro-5-en-3β-yl-α-L-rhamnopyranosyl-(1->2glu)-O-α-L-rhamnopyranosyl-(1->4glu)-β-D-gluco-pyranose](33%), and their corresponding di- and monoglycosides (34%). All the glycosides contain the same aglycone, solasodine. This mixture of glycosides which includes solasonine and solamargine is commonly referred to as BEC. The structures of solasonine and solamargine are shown below:

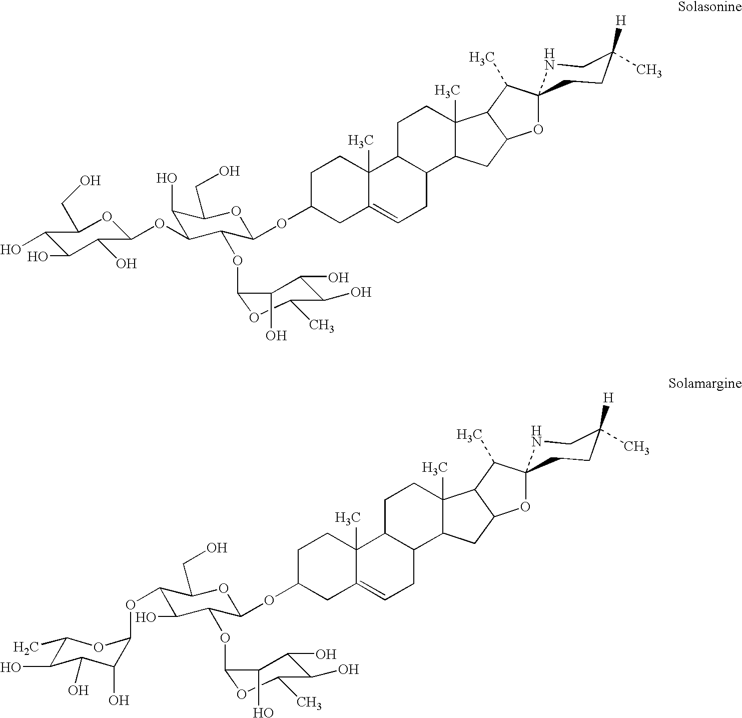

The anti-cancer properties of BEC has been studied in vivo with mice inoculated with murine sarcoma 180 and in cell culture studies. BEC was observed to selectively destroy tumor cells relative to normal cells. The efficacy and specificity of BEC was also observed to be dependent upon the type of tumor. These observations were attributed to the presence of endogenous endocytic lectins or EEL's present on the membranes of those cells observed to be susceptible to BEC. EEL's are endogenous receptors which have been reported to be expressed during human embryogenesis and carcinogenesis. Interaction of the EEL with a molecule or ligand for which it is a receptor results in internalization of the EEL and bound ligand.

It is believed that the tumor cells susceptible to BEC have EEL receptors specific for the glycoside portion of the glycoalkaloids in BEC. These EEL's selectively recognize and bind the sugar moiety of the glycoalkaloid. The glycoalkaloid is subsequently internalized and the result is destruction of the cell. The mechanism of cell destruction is believed to be by cell lysis.

That there is an EEL specific for the glycoside moiety of the glycoalkaloid is supported by a number of observations. First, the aglycone, solasodine, when administered at levels at which BEC is effective is ineffective against tumor cells. The sugar portion of the glycoalkaloid on its own is also ineffective. Second, competition studies have also shown that at least a three fold molar excess of the sugar rhamnose is required to inhibit the cytotoxicity of BEC. Solamargine contains two molecules of rhamnose and solasonine, one molecule. It should be noted that rhamnose is a plant sugar and is rarely found in mammalian cells. Thus, it is unlikely that normal mammalian cells have a receptor for rhamnose. The aforementioned competition studies were conducted on mice with murine sarcoma 180. Untreated mice died in 2-3 weeks. Four doses of 8 mg/kg BEC given on consecutive days resulted in survival of virtually all animals. Five mg rhamnose/kg decreased the survival to 75%, 10 mg rhamnose/kg decreased the survival to 50% and 15 mg rhamnose/kg decreased the survival to 42%. Similar concentrations of rhamnose were observed to have no effect on S180 activity in the absence of BEC.

Acute toxicity studies for BEC were also carried out in mice. These studies showed that for single intraperitoneal (ip) doses of BEC, the LD₅₀ was 30 mg/kg. For administration of 14 daily single ip doses, the LD₅₀ for mice was 10 mg/kg. In contrast it was shown that the ED₅₀ (quantal effective level for 50% of the population after given a single dose) for a single dose of BEC was 9 mg/kg. With 3 and 4 administrations at 9 mg/kg BEC to mice with Sarcoma 180, greater than 95% of the mice were rendered cancer free for the remainder of their life span. The quantal effective levels (ED₅₀) of BEC for single administrations were similar to the lethal levels (LD₅₀) for multiple administrations of 10 mg/kg at 14 daily ip doses. BEC has also been observed to be effective for melanoma and ovarian tumor cells grown in cell culture. The therapeutic index (LD₅₀/ED₅₀) for these cell culture trials was about 3.

It can be seen that a disadvantage of BEC is the toxicity of the preparation when administered at the very high levels required to successfully treat internal cancers. It would therefore be desirable to obtain a glycoalkaloid composition which is more effective than BEC for the treatment of cancers.

The above toxicity studies also provide further support for the EEL mediated activity of glycoalkaloids against cancer cells. Mice with advanced cancer activity could tolerate up to three times the LD₁₀₀ of BEC. This could be explained by selective absorption of BEC by the cancer cells which were present in abundance. Thus the bioavailablity of BEG to normal cells could be reduced. These toxicity studies also showed that ingestion of BEC into normal cells can occur by routes other than selective recognition by EEL's at high concentrations. For example, BEC at high concentrations may diffuse through the plasma membrane of the cell.

Treatment of premalignant and malignant skin lesions with BEC in humans has also been studied. Topical application of BEC has been observed to be effective for the treatment of lesions consisting of keratosis, basal cell carcinoma and squamous cell carcinoma. Creams containing 10% and 0.005% BEC when applied topically showed complete clinical and histological regression when applied twice daily over treatment periods of up to about three months. Although the final result of the 10% and 0.005% treatments were comparable in relation to regression of the disease, the duration of the treatment with the 0.005% BEC required for regression of the lesions was considerably longer than for the 10% BEC. Typically the treatment period required for the low concentration of BEC was about 13 to 14 weeks.

The extended duration of the treatment for the low concentration BEC formulations has a number of disadvantages. First there is a difficulty with patient compliance. For optimum effectiveness, the BEC formulation must be applied at regular intervals, typically twice a day, until clinical regression is observed. Many patients find it difficult to comply with such a regime for up to 14 weeks. During this period, patients may experience an unacceptable amount of pain due to high salicyclic acid concentrations. Further, during treatment, as the affected cells undergo lysis, the lesions ulcerate and should be covered by a dressing. From a cosmetic and patient comfort perspective it would be desirable to be able to reduce the duration of treatment. Although such a reduction can be achieved by increasing the dose of BEC, this is undesirable in view of the toxicity of BEC. Still further, as large amounts of plant product are required to produce small amounts of BEC, the 10% BEC preparation is quite expensive to produce. It is therefore desirable to be able to obtain a low dose glycoalkaloid composition for the treatment of skin conditions, which results in clinical regression in a relatively short period of time and is also cost effective.

### OBJECT OF THE INVENTION

It is therefore an object of the present invention to provide an improved glycoalkaloid composition for interaction with target cells and which may be used for the treatment of cancer and tumors in mammals and which may at least partially overcome the above disadvantages or provide the public with a useful choice.

### SUMMARY OF THE INVENTION

Glycoalkaloids can undergo degradation in which the glycoside moiety or a saccharide unit thereof is cleaved from the alkaloid. Where the glycoside moiety of the glycoalkaloid includes two or more saccharide units, there are a number of possible degradation products including free sugars such as monosaccharides, disaccharides and trisaccharides; the aglycone and mono and diglycosides.

It has been surprisingly and unexpectedly discovered that the efficacy of a glycoalkaloid formulation against cancer, other abnormal cells or other target cells having EEL's can be inhibited by very low amounts of free sugars which may be produced as a result of degradation of the glycoalkaloid.

In the present specification and claims, the term “free sugars” refers to any sugar such as a mono, di, trisaccharide, oligosaccharide or polysaccharide or derivative thereof which is not bound to an alkaloid.

According to a first broad form of the invention, there is provided a medicinal composition comprising at least one compound which can interact with a target cell, the at least one compound being a glycoalkaloid of the general formula I:

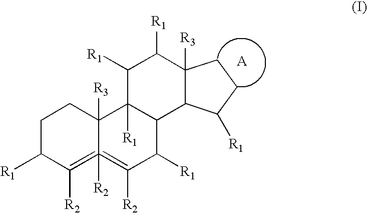

wherein:

either one of the dotted lines represents a double bond, and the other a single bond, or both represent single bonds;

A: represents a radical selected from the following radicals of general formulae (II) to (V):

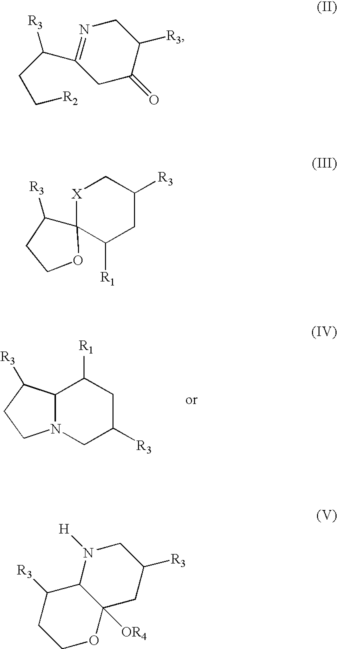

each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, alkyl and R⁴O-alkylene; each of R⁴ is a radical separately selected from the group consisting of hydrogen, carbohydrate and a carbohydrate derivative;“X” is a radical selected from the group comprising —CH₂—,—O— and —NH₂—;

wherein the compound includes at least one R⁴ group in which R⁴ is a carbohydrate or a derivative thereof; together with a pharmaceutically acceptable carrier, adjuvant, excipient and/or diluent, wherein the composition is essentially without free sugars of the type which inhibit the interaction between the at least one glycoalkaloid and a target cell.

Preferred carbohydrate radicals R⁴ are glyceric aldehyde; glycerose; erythrose; threose; ribose; arabinose; xylose; lyxose; altrose; allose; gulose; mannose; glucose; idose; galactose; talose; rhamnose; dihydroxyactone; erythrulose; ribulose; xylulose; psicose; fructose; sorbose; tagatose; and other hexoses (C₆H₁₂O₆); heptoses (C₇H₁₄O₇); octoses (C₈H₁₆O₈); nanoses (C₉H₁₈O₉); decoses (C₁₀H₂₀O₁₀); deoxysugars with branched chains (eg. apiose, hamamelose, streptose, cordycepose, mycarose and cladinose); compounds wherein the aldehyde, ketone or hydroxyl groups have been substituted (eg. N-acetyl, acetyl, methyl, replacement of CH₂OH); sugar alcohols; sugar acids; benzimidazoles; the enol salts of the carbohydrates; saccharinic acids; sugar phosphates.

The more preferred compounds are solasonine, solamargine, solanine and tomatine.

Other preferred compounds of the general formula (1) are solanocapsine and 26-aminofurostane.

It will be appreciated that the various compounds referred to throughout this specification may be chiral and the present invention relates both to the individual stereoisomers and to any mixtures thereof including mixtures of enantiomers and/or diastereoisomers.

A preferred composition of the present invention is a solasodine glycoside composition which includes solasonine, solamargine and their di and monoglycosides in the same or similar proportion as the aforementioned BEC.

The composition of the present invention typically comprises naturally occurring glycoalkaloids extracted from a plant source. Generally, the plant extract is treated to remove essentially all of any free sugars which can inhibit the efficacy of the glycoalkaloids prior to formulation of the composition of the present invention. Although it may be possible that one or more free sugars do not inhibit the efficacy of the glycoalkaloids and do not need to be removed, typically all of the free sugars will be removed from the plant extract.

According to a further broad form of the invention there is provided a method of preparing a glycoalkaloid preparation comprising at least one glycoalkaloid according to formula I, as hereinbefore defined, the method including extracting the at least one glycoalkaloid from a suitable plant material to form a crude extract, and removing essentially all free sugars from the crude extract.

The crude extract may be obtained by any suitable method. When the plant material is Solanum_{—}Sodomaeum a preferred method is to extract coarsely ground plant material with acetic acid. The extract is filtered and the pH adjusted to about 9 to 10 to obtain a precipitate. The precipitate may be dissolved in acetic acid and re-precipitated at high pH. The precipitate is typically further extracted with ethanol to provide the solasodine glycoside mixture or BEC as a semicrystalline powder.

The free sugars may be removed from the plant extract by any suitable method. A preferred method is to wash the crude extract in water or other suitable solvent. Generally, the free sugars are removed to below detectable limits or are at least removed to a level below which an inhibitory effect can be detected. Generally, the composition of the present invention is essentially without all free sugars. However, it will be appreciated that free sugars which do not inhibit the cytotoxicity of the glycoalkaloids may be present.

The composition of the present invention may also be formulated from a synthetic glycoalkaloid or a mixture of glycoalkaloids. In this case, the synthetic glycoalkaloids would typically be treated prior to formulation of the composition to remove any sugars present as a result of glycoalkaloid degradation.

The glycoalkaloids in the composition of the present invention may also be obtained from chemical modification of naturally occurring glycoalkaloids. In this case, the naturally occurring sugar moiety of the glycoalkaloid can be modified by removing or adding a saccharide unit or units. Suitable methods of carbohydrate modification are known and include chemical or enzymatic hydrolysis. Alternatively, the sugar moiety may be completely removed and replaced with a different sugar moiety. An advantage of such modification of the sugar group of a glycoalkaloid is to be able to modify the efficacy or selectivity of that glycoalkaloid towards a desired target cell.

It is believed that the mode of action of glycoalkaloids against target cells is by EEL mediated endocytosis in which an EEL recognizes the sugar moiety of the glycoalkaloid and subsequent internalization of the EEL and glycoalkaloid. Thus, by identifying those sugars which can be recognized by receptors on a desired target cell, a modified glycoalkaloid may be derived which is specific to that receptor. In this way a glycoalkaloid can be designed to target a desired cell type.

The products of glycoalkaloid degradation may also include the aglycone. Preferably, any aglycone is also removed prior to formulation of the therapeutic compositions of the present invention. Removal of the aglycone may be conducted by any suitable means and is typically removed by solvent extraction. Suitable solvents include the chlorinated hydrocarbon solvents and chloroform is particularly preferred.

Under normal storage conditions, some degradation of glycoalkaloids in a pure or semi-pure crystalline or semicrystalline form can occur. Thus, it is preferred, that where storage has occurred, the aforementioned sugar removal and if desired aglycone removal of stored glycoalkaloid be conducted immediately prior to formulation of the therapeutic compositions of the invention. Typically the composition is stabilized against glycoalkaloid degradation. Typically, the composition is acidic and preferably includes acetic or lactic acid. The acidic conditions minimize degradation to produce free sugars.

Alternatively, sugar free glycoalkaloid preparations including the crystalline form may be prepared and then stored under stable conditions prior to formulation of the therapeutic composition of the present invention. The sugar free preparation may be stored in an acidic solution and/or at low temperature.

According to a further broad form of the present invention, there is provided a method of preparing a therapeutic composition which comprises a therapeutically effective amount of at least one glycoalkaloid according to formula I, as hereinbefore defined, the method including obtaining at least one glycoalkaloid, removing any free sugars from the glycoalkaloid and mixing the glycoalkaloid with a pharmaceutically acceptable stabilizer.

The amount of the glycoalkaloid present in the therapeutic composition of the present invention may depend on the dose rate, patient, the type of condition being treated and in the case of a tumor the type, size and position of the tumor to be treated. In the preferred composition which includes solasodine glycosides, a typical composition for the treatment of skin tumors would typically include between about 5 to about 0.001%, preferably about 0.005% solasodine glycosides.

The therapeutic composition of the present invention may be used in the treatment and control of conditions which may be treated or controlled by selective cellular destruction or modification. Such uses include the treatment or control of cancer, contraception, termination of pregnancy, removal of pathogenic organisms and removal of abnormal cellular growth.

According to a further broad form of the present invention there is provided a method for the treatment or control of cancer, contraception, termination of pregnancy, removal of pathogenic organisms and removal of abnormal cellular growth in a mammal requiring such treatment, the method comprising administering to the mammal an effective amount of a medicinal composition or preparation of the present invention.

The medicinal composition of the present invention may be formulated in any suitable manner including injectable compositions, tablets, suppositories, capsules and topical formulations. In a preferred formulation for the treatment of skin tumors or lesions, the formulation is a cream for topical administration or an injectable formulation. In the case of an internal cancer or sarcoid, the composition may be an injectable formulation for intraperitoneal or intralesional injection.

Typically, the injectable composition is administered in an amount of between about 50 to about 200 mg of sugar free glycoalkaloid composition per kg of tumour. Animal and human studies (as illustrated in the following examples) show that successful treatment of some tumors and cancers may be accomplished with as few as two to four injections. The injection may be given at one, two or three daily intervals, preferably the treatment is given twice, at day 1 and day 3. Treatment by injection may also be given in association with topical administration if desired or considered necessary.

It has also been surprisingly discovered that the therapeutic composition of the present invention may also be used to diagnose skin conditions before such conditions can be detected by visual inspection.

Such diagnosis may be carried out by broadly applying a composition of the present invention to an area of skin to be tested. The composition is left on the skin for a pre-determined period of time. During this time, any abnormal cells are selectively destroyed. This produces a detectable inflammation of the affected areas which may then be identified and treated.

According to a further broad form of the present invention there is provided a method of diagnosing a skin condition in a mammal, the skin condition being caused by the presence of abnormal cells, wherein the method includes applying an effective amount of a composition or preparation of the present invention to an area of skin to be diagnosed, leaving the composition on the skin for a pre-determined period of time, removing the composition and detecting any change to any areas of skin.

The diagnostic method of the present invention is particularly suitable for diagnosing skin conditions of humans. Typical conditions which may be diagnosed include Keratoses, basal cell carcinomas, squamous cell carcinomas, melanomas or other skin cancers.

A particularly preferred diagnostic composition is a solasodine glycoside mixture having about the same glycoside composition as BEC but without free sugars or the aglycone, solasodine. In trials conducted by the present inventor it has been observed that the normal healthy skin tissue is unaffected by the composition. This demonstrates the selectivity of glycoalkaloids for abnormal cells.

This method of diagnosis allows skin conditions to be detected and treated at an early stage, typically before the condition produces visible skin lesions.

It should be appreciated that such a method of diagnosis would not be possible with conventional skin treatment compositions which adversely affect all cells. A further advantage of such specificity is that during application, should the composition be inadvertently applied to a patients' healthy skin, the healthy skin will not be damaged. This does not occur with conventional skin treatment where care must be exercised to avoid contact with healthy skin.

Further, in view of the suprisingly improved efficacy of the present invention in treatment of skin conditions, the diagnosis can be conducted using very low concentrations of solasodine glycosides.

### BRIEF DESCRIPTION OF THE DRAWINGS

FIGS. 1 and 3 and FIGS. 2 and 4 illustrate HPLC spectra for unwashed and washed BEC respectively.

FIGS. 5 to 8 illustrate a sarcoid tumor in a horse, before (FIG. 5), during (FIGS. 6 and 7) and after (FIG. 8) treatment with a preferred composition of the present invention.

FIGS. 9 to 12 illustrate the stages in the treatment of a horse with a penile sarcoma before (FIG. 9) during (FIGS. 10 and 11) and after (FIG. 12) treatment with a preferred composition of the present invention.

FIGS. 13 to 15 illustrate stages in the treatment of a human with squamous cell carcinoma before (FIG. 13) and during (FIGS. 14 and 15) treatment.

### BEST MODE

The present invention will now be described with reference to the following non-limiting examples.

### EXAMPLE 1

A sugar free solasodine glycoside preparation was prepared according to the following:

50 kg Solanum Sodomaeum berries are put through commercial meat mincer (fitted with 1.HP electric motor 1425 rpm) with a sieve size of 3 mm.

The slurry is diluted with 3% acetic acid (pH 2.5) (food grade) to a volume of 200 L. This semi-solid solution is treated with a Silverson homogenizer for 15 minutes. Mixing is continued for another 4 hours using a SS rod with arms mixer at room temperature at 30 rpm (Flamingo CMG 0.75 kw variable speed control meter).

The solution is allowed to stand overnight without mixing. The solution is subsequently filtered through a muslin cloth. The filtrate is then subjected to a flow through centrifuge (3.5 HP) at 1455 rpm. The clear filtrate is heated to 50° C. in a stainless steel double jacketed bowl. Concentrated ammonia (L R Grade) is added until pH≈10. A precipitate is observed. The precipitate is allowed to settle and cool (approx. 24 hrs). The supernatant is carefully decanted. The precipitate is dissolved in 25 L 3% aqueous acetic acid. The solution is centrifuged through flow through centrifuge as above. The supernatant is collected in an SS double jacketed bowl and heated to 50° C. with continuous stirring (30 rpm, 30 min).

The glycoalkaloids are re-precipitated by the addition of concentrated ammonia solution until pH≈10. The solution is allowed to cool and the precipitate is allowed to settle (approx. 24 hrs). The supernatant is carefully decanted and the precipitate is washed with 50 L water and allowed to settle for 24 hrs as before. The supernatant is decanted and this procedure is repeated four times.

The precipitate is finally dissolved in 10 L alcohol at 75° C. and filtered whilst hot through Whatman No. 1 filter paper. The supernatant is dried at 50° C. This yields a fine, semicrystalline powder. The yield is 505 g which is 1.01%.

Any aglycone, solasodine, is removed by washing the extract in chloroform. The solasodine is soluble in the chloroform phase and the sugars are soluble in the aqueous phase. The glycoalkaloids remain insoluble under all these conditions.

### EXAMPLE 2

Cream formulations were prepared from the sugar free solasodine glycoside preparation from Example 1 as follows:

| | Percentage | |
|---|---|---|
| Active Ingredient | Composition | Function |
| Solasodine Glycosides | 0.005% w/w | Antineoplast |
| (BEC) | | |
| Other Ingredients | | |
| Cetomacrogol | 15.0% w/w | Emulsifying agent |
| Emulsifying wax | | |
| White soft paraffin | 10.0% w/w | Cream base |
| Liquid paraffin | 10.0% w/w | Cream base |
| Salicylic acid | 10.0% w/w | Keratolytic |
| Urea | 5.0% w/w | Keratolytic |
| Propylene glycol | 5.0% w/w | Emollient |
| Chlorocresol | 0.1% w/w | Preservative |
| Acetic or lactic acid | qs | Solvent |
| Purified water | qs | Solvent/Cream base |

Emulsifying wax, white soft paraffin, liquid paraffin, propylene glycol and water were used to provide a cream base of a suitable consistency and viscosity. Chlorocresol was included in the formulation as a preservative. Salicylic acid and urea were included as keratolytic agents and were considered to be excipients in the cream formulation because their primary function was to enhance the bioavailability of the active ingredient by clearing tissue from around the tumor, thus allowing a higher concentration of the active ingredients to reach the tumor. (The International Pharmaceutical Excipients Council definition of an excipient includes substances which are included in a drug delivery system to protect, support or enhance stability, bioavailability or patient acceptability and to enhance any other attribute of the overall safety and effectiveness of the drug during storage or use).

Acetic or lactic acid was present in the final formulation because a 3% solution of acetic acid is used as the solvent for the active ingredients during the manufacturing process.

### EXAMPLE 3

White soft paraffin, liquid paraffin and cetomacrogol emulsifying wax were weighed into a sanitized stainless steel container (“Phase A”). This mixture was gently heated on a low burner until the temperature reached 70° C.

Purified water at 70° C., urea, chlorocresol and propylene glycol were added to a suitable stainless steel container and mixed for 2 minutes using a Silverson mixer (“Phase B”).

The melted Phase A was slowed added to Phase B and thoroughly mixed using a Silverson mixer or follow-through homogenizer. The mixture was allowed to cool to about 50° C. and then the salicylic acid was added.

The freshly washed solasodine glycosides were dissolved in acetic is acid or lactic acid solution and added to the cream, with mixing to ensure even dispersion. The cream was allowed to cool to room temperature with occasional mixing to ensure an even, smooth texture.

The formulated cream had the following specifications:

| | |
|---|---|
| Description | Smooth, white or slightly off-white cream |
| BEC assay | 0.0046-0.0054% |
| Salicylic acid assay | 9.5-10.5% |
| pH | Less than 3 |

### EXAMPLE 4

The solasodine glycoside preparation from Example 1 and creams from examples 2 and 3 were analyzed for hydrolysis products by MS and HPLC according to the following procedure.

Sample Preparation:

Standard was prepared in 50% CH₃CN/H₂O at 1 mg/ml and 100 ug/ml. Cream was prepared by dissolving 100 mg cream in 2 ml methylene chloride, after centrifugation 100 ul of the aqueous phase was removed and made up to 1 ml with methanol.

HPLC Conditions:

A Waters Alliance system was used consisting of a 2690 separations module, and 996 diode array detector. A Micromass Waters Platform LCZ mass spectrometer was interfaced and the whole system was controlled by MassLynx chromatography software.

Solvent:

Isocractic analysis was performed with 75% CH₃CN/H₂O

Gradients were run from 80% CH₃CN to 50% CH₃CN over 7 minutes

Flow Rate was 1.0 or 0.5 ml/min

UV Detection was from 205 nm to 320 nm (205 nm was extracted for alkaloids and 254 for salicylic acid and chlorocresol)

Mass Spec full scans from 400-900 m/z were used for TIC chromatograms and single channels of 869 and 885 were used for quantitation.

Full detailed conditions including cone voltages are attached.

Column

High Performance Carbohydrate column (4 um) 0.46*25 cm was used.

Results

The standard gave three peaks corresponding to Solasonine, Solamargine and an unidentified peak at mass 722. Some smaller peaks were also observed but no indication of mass 414 consistent with the aglycone, hence no obvious hydrolysis of the samples. The cream showed both actives as well as propylene glycol, salicylic acid and chlorocresol. MS results were approximately 1000 times more sensitive than UV.

### EXAMPLE 5

HPLC studies were also conducted on BEC preparations obtained by conventional extraction procedures (i.e. without any washing steps) and stored for a period of up to about 6-8 months. HPLC analysis was conducted on the stored BEC both before and after washing to remove free sugars and solasonine.

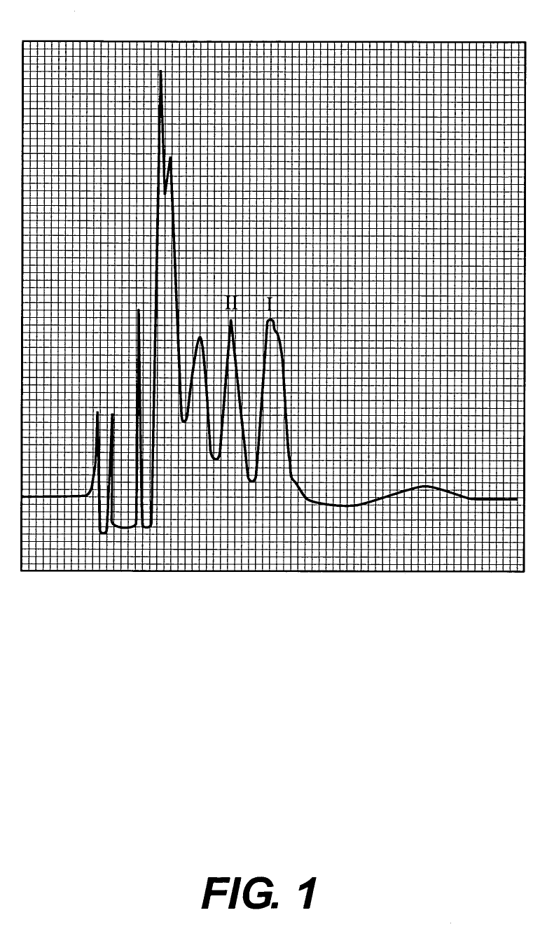

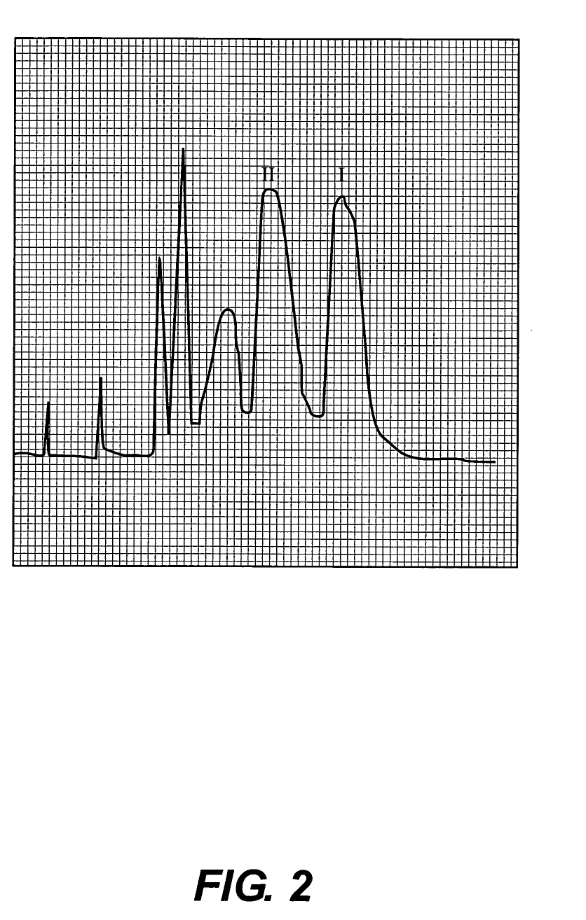

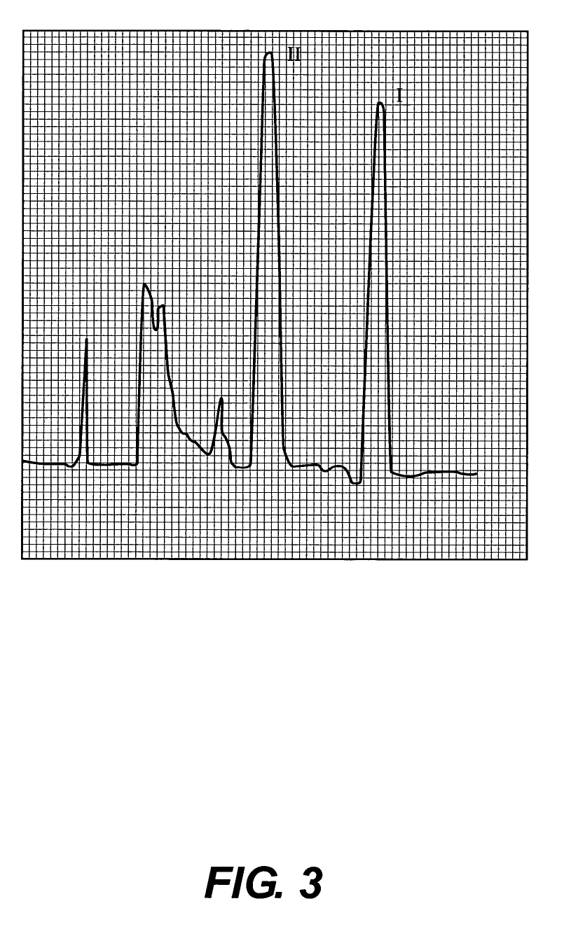

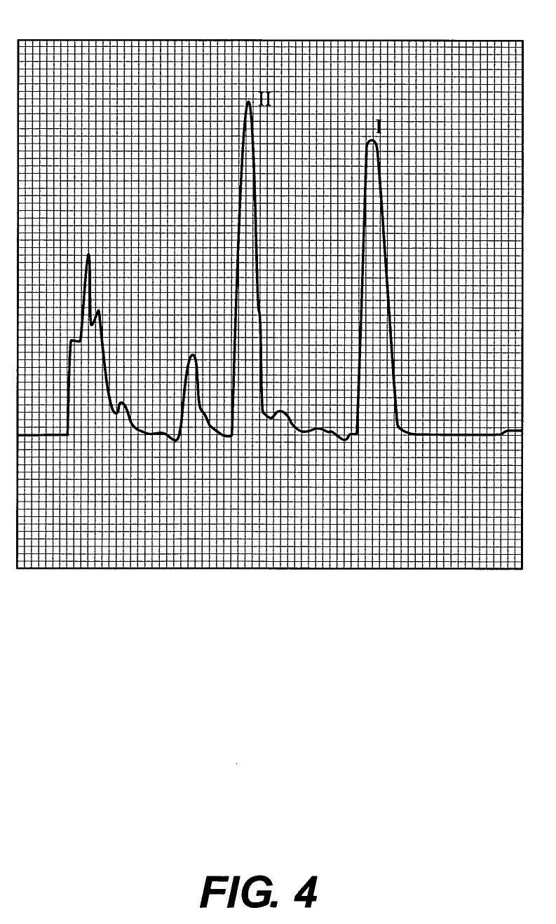

FIGS. 1 to 4 illustrate HPLC spectra of unwashed (FIGS. 1 and 3) and washed (FIGS. 2 and 4) BEC respectively.

Compounds marked as I and II had the same elution times as solasonine and solamargine standards.

FIGS. 1 and 2 show that the unwashed BEC includes a number of further peaks. By comparison with FIGS. 3 and 4, it can be seen that these peaks have been removed or significantly decreased upon washing with water and chloroform. These further peaks have been assigned to the various sugar degradation products of BEC.

The compounds represented by peaks I and II have increased in height relative to the remaining peaks.

It can be seen that BEC undergoes degradation under normal storage conditions. These degradation products may be removed by washing the stored BEC with water and chloroform.

### EXAMPLE 6

A BEC extract was obtained from Solanum Sodomaeum according to conventional extraction procedures. The degradation of the BEC extract was estimated by measuring the change in solamargine and solasonine levels over time. Although degradation of BEC could also be measured by an increase in sugar levels, in practice HPLC analysis for solamargine and solasonine allowed a more quantative analysis to be conducted and was therefore chosen for this study.

The results are illustrated in the following Table.

Degradation (%) mean ± .S.D.

| Time/Years | Solamargine | Solasonine |
|---|---|---|
| 0 | 0 | 0 |
| 0.5 | 4 ± 4 | 5 ± 5 |
| 1 | 8 ± 5 | 10 ± 5 |
| 2 | 12 ± 7 | 15 ± 8 |
| 3 | 15 ± 8 | 19 ± 9 |
| 4 | 19 ± 10 | 21 ± 10 |
| 5 | 22 ± 10 | 23 ± 11 |

It can be seen that degradation of the solasodine glycosides of the BEC occurs over time. The effectiveness of cream formulations prepared from this BEC was observed to decrease with the time the BEC was stored prior to formulating the cream. This decrease in efficacy resulted in an increase in the duration of treatment required for regression of skin conditions treated by the cream.

It will be appreciated that even after 5 years the relative amounts of free sugars produced by degradation of solamargine and solasonine are present in relatively low amounts. Any inhibition at these low levels could not have been predicted from the observation that a large molar excess of rhamnose in the aforementioned studies. It should also be noted that the decrease in efficacy observed with stored BEC is inconsistent with what would be predicted from the small decrease in concentration of the active agents, solamargine and solasonine.

### EXAMPLE 7

The survival of mice with sarcoma 180 when treated with varying doses of 7 mg and 8 mg unwashed and washed BEC/kg.

Washed BEC was prepared according to Example 1 and administered immediately after preparation.

Unwashed BEC was prepared in a similar manner but was not washed prior to use. The unwashed crystalline BEC was stored under ambient conditions for about four weeks prior to administration to the mice. By reference to the degradation studies provided in the previous example, the % degradation over the four weeks can be estimated to be between less than 4 to 5% for solamargine and solasonine respectively.

Although, this degree of degradation may be considered to be negligible, it can be seen that there is a significant decrease in efficiency. Thus, BEC should be washed prior to formulation even after storage for even short periods of time (such as about four days).

12 mice with sarcoma 180 were treated with 7 mg/kg doses given on consecutive days. The results are shown in the following table.

| | | No. of | | Animals |
|---|---|---|---|---|
| Compound | Dose | Doses | Survival Time | Survived/Treated |
| | | | 20.9 ± 5.6 | 0/12 |
| BEC | 7 | 1 | 20.9 ± 6.0 | 0/12 |
| Unwashed | | | | |
| | 7 | 2 | 29.1 ± 6.6 | 2/12 |
| | 7 | 3 | 37.5 ± 16.2 | 4/12 |
| | 7 | 4 | 42.0 ± 17.1 | 6/12 |
| BEC Washed | 7 | 1 | 25.3 ± 6.1 | 0/12 |
| | 7 | 2 | 44 ± 14.2 | 7/12 |
| | 7 | 3 | 53.0 ± 10.0 | 11/12 |
| | 7 | 4 | 56.0 | 12/12 |
| BEC | 8 | 1 | 20.9 ± 5.5 | 0/12 |
| Unwashed | | | | |
| | 8 | 2 | 30.1 ± 15.8 | 4/12 |
| | 8 | 3 | 48.0 ± 16.2 | 11/12 |
| | 8 | 4 | 53.0 ± 12.6 | 11/12 |
| BEC Washed | 8 | 1 | 38.3 ± 10.2 | 7/12 |
| | 8 | 2 | 55.1 ± 6.8 | 11/12 |
| | 8 | 3 | 56.0 | 12/12 |
| | 8 | 4 | 56.0 | 12/12 |

Each value is the mean ±S D obtained in groups of twelve tumour —bearing mice treated intraperitoneally 0.5 h after tumour implantation (5×10⁵ cells/mouse).

^{a} The criterion of survival was taken as 56 days because it was shown that if the treatment was effective against sarcoma 180 for this period, the mice then had a normal life span (approximately 3 years).

^{b} Doses given on consecutive days.

^{c} Animals surviving after eight weeks.

It can be seen that the % survival for animals treated with the washed BEC was superior when compared with animals treated with an equivalent dose of unwashed BEC. For example, the % survival rate for four doses of unwashed BEC is 50% as compared with 100% for washed BEC.

### EXAMPLE 8

The effect of washed and unwashed BEC or human ovarian cancer cells was compared. The washed and unwashed BEC were prepared as described for Example 8.

Cells (5×10⁴) were transferred (200 μl chamber of a microscope slide (Lab Tek Miles Scientific). Controls received 50 μl HIFCS/TCM and experimental chambers 50 μl of solasodine glycosides (BEC) 1.5-3.8 μM/L, washed and unwashed after 7-h preincubation and incubated for a further 17 h and 3-15.3 μM/12L h preincubation and incubated for a further 3h. Similarly, the cells were treated with the aglycone solasodine 19.4-96.8 μM/L. The cells were fixed and examined by the Papanicolaou method.

The results are shown in the following table.

| BEC unwashed | | BEC washed | |
|---|---|---|---|
| μg/mL | % survival | μg/ml | % survival |
| 0 | 100 | 0 | 100 |
| 1 | 92 | 1 | 80 |
| 2 | 95 | 2 | 30 |
| 3 | 92 | 3 | 10 |
| 4 | 77 | 4 | 3 |
| 5 | 15 | 5 | 1 |
| 6 | 3 | 6 | 1 |
| 8 | 1 | 8 | 1 |
| 10 | 1 | 10 | 1 |

| Solasodine | |
|---|---|
| μg/ml | % survival |
| 0 | 100 |
| 3 | 100 |
| 6 | 100 |
| 9 | 100 |
| 12 | 100 |
| 15 | 100 |
| 18 | 100 |
| 24 | 100 |

These results again illustrate the surprisingly superior efficiency of the washed BEC.

### EXAMPLE 9

A patient with no visible lesions on the face had a cream as prepared in Example 2 applied to the skin of the face. The cream was left for 30 minutes before being washed away. The patient's face was then examined. Areas of redness were noted which were identified as pre-malignant or malignant skin lesions in the very early stages of development. The affected areas of skin were subsequently treated with the same cream.

### EXAMPLE 10

A human patient was diagnosed with an intra epithelial penile tumour.

The prognosis was that no treatment was available and that amputation was the only option. The patient commenced treatment with the cream as prepared in Example 2 and was applied to the tumour twice daily. Necrosis of the tumour was observed to occur shortly after treatment commenced. Within six weeks, the patient was observed to be free of the tumour.

### EXAMPLE 11

The use of a preferred composition of the present invention was trialed on solid tumors in animals and humans as follows:

Formulation: A sugar and aglycone free solasodine glycoside preparation which was prepared according to Example 1 in DMSO. DMSO is used for its aprotic characteristics and because when pure is sterile.

| | |
|---|---|
| Models: | Horses, Dogs, Humans. |
| Lesions: | Solid Sarcoids and squamous cell carcinoma |
| | (SCC). |

Procedure: The approximate weight of the sarcoid or SCC is assessed then 100 mg of the sugar and aglycone free solasodine glycosides preparation of Example 1 (100 mg/ml DMSO of stock solution) is injected intralesionally to 1 kg tumor weight. Two days later this procedure is repeated.

Results: At day 2 after the first injection, massive necrosis is observed. Two weeks later ablation of tumor is achieved.

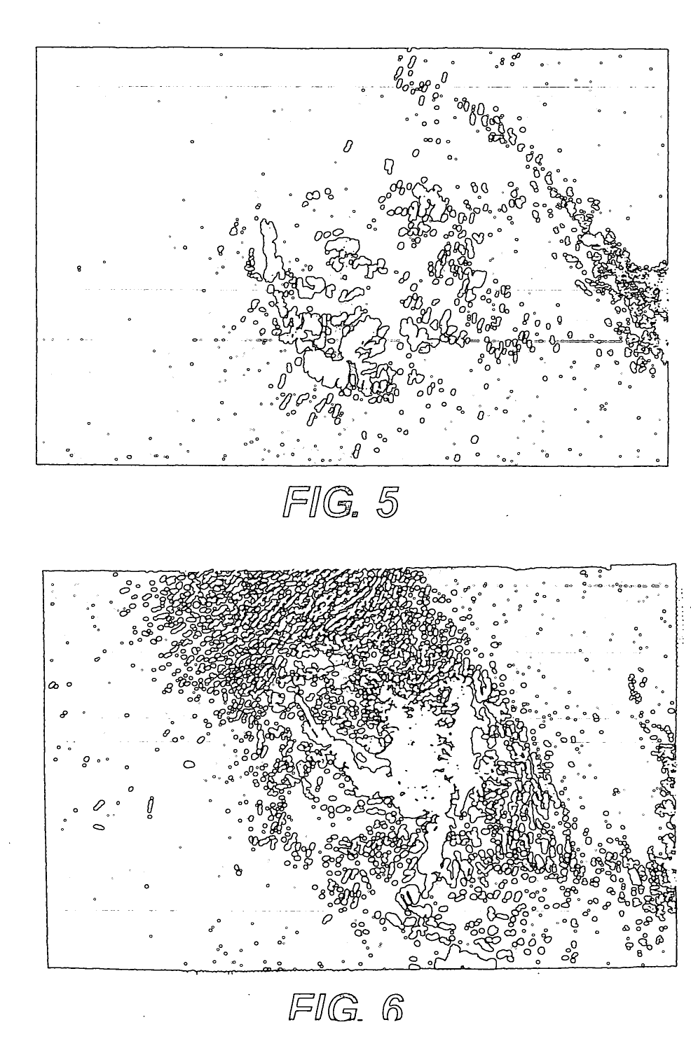

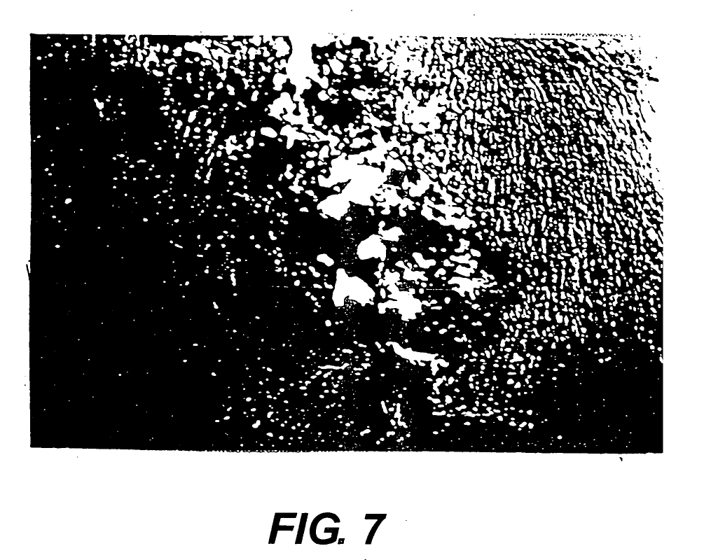

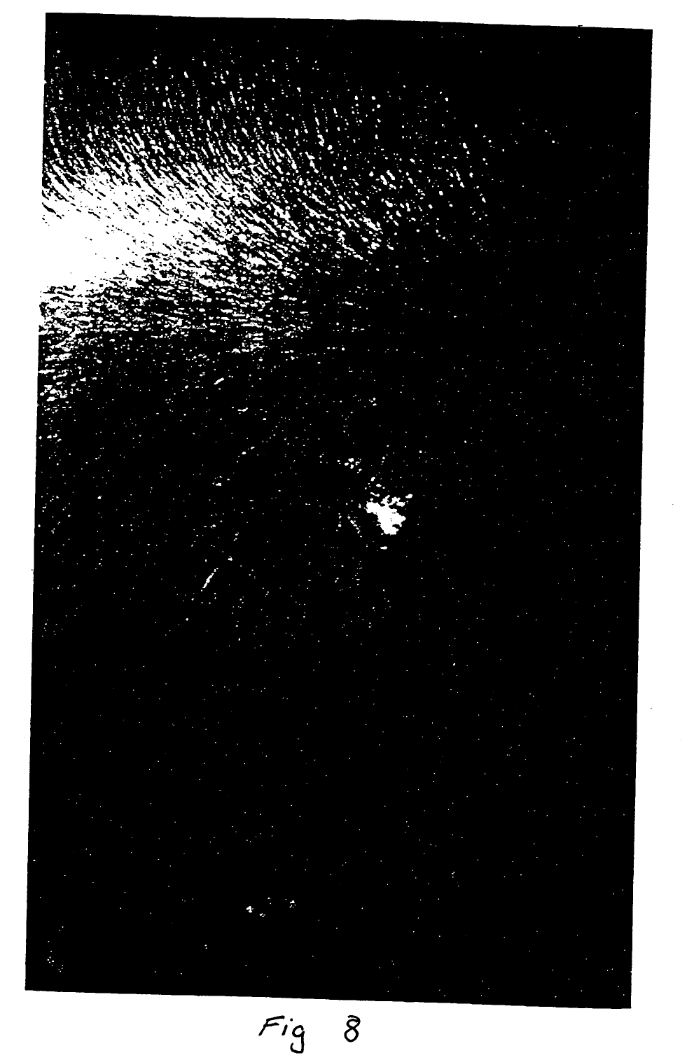

FIGS. 5, 6, 7 and 8 show an example of a sarcoid tumor in a horse, before, during and after treatment. FIG. 5 illustrates the sarcoma before treatment. FIGS. 6 and 7 show the sarcoma after injection with the above composition. Necrosis of the sarcoma can be seen. FIG. 4 shows that the sarcoma has fully regressed after treatment.

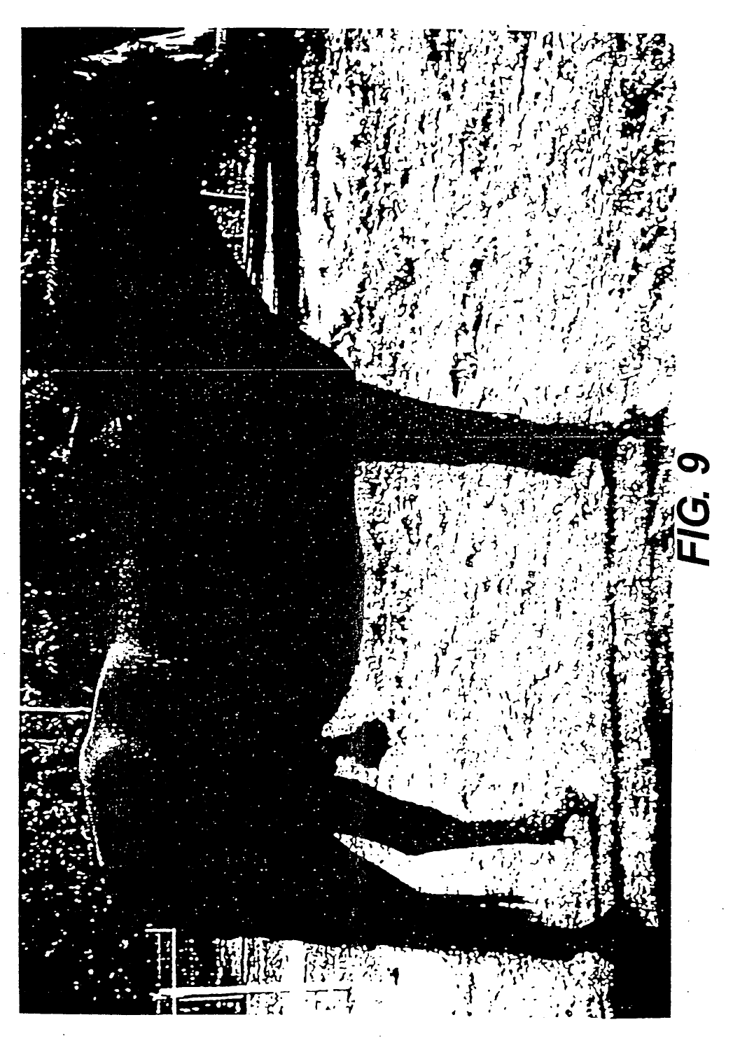

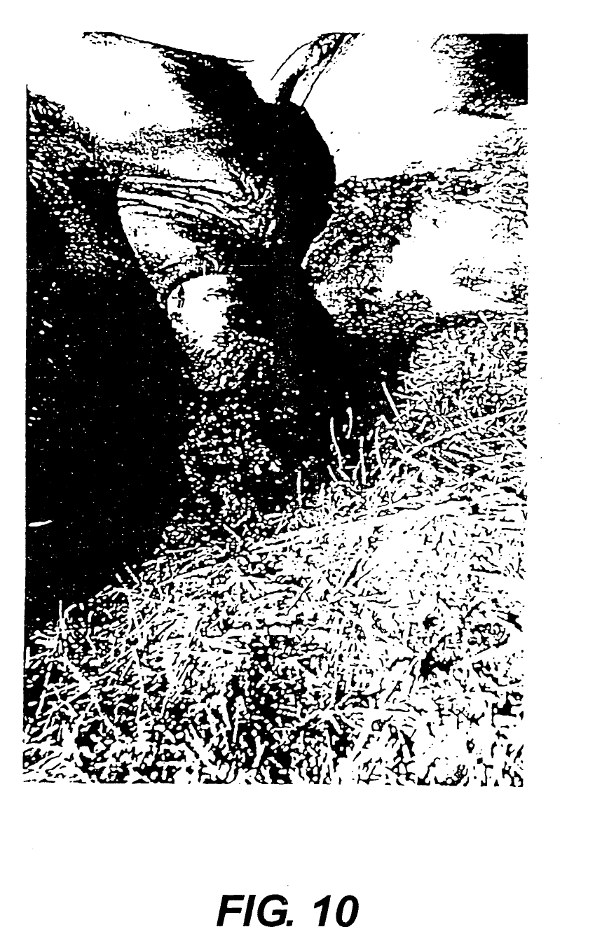

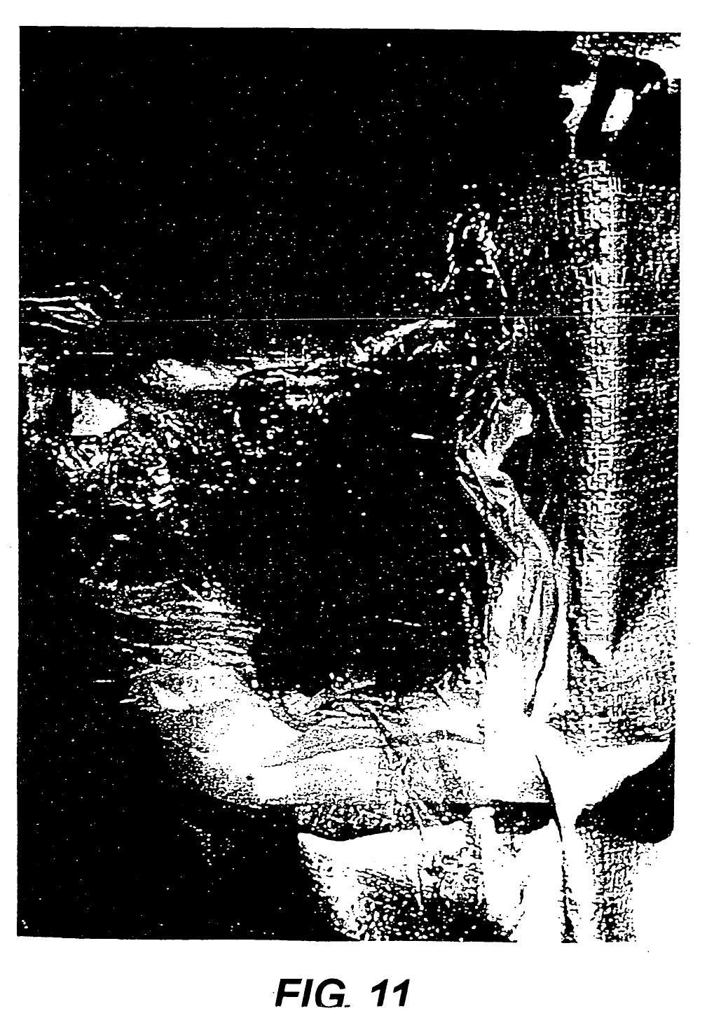

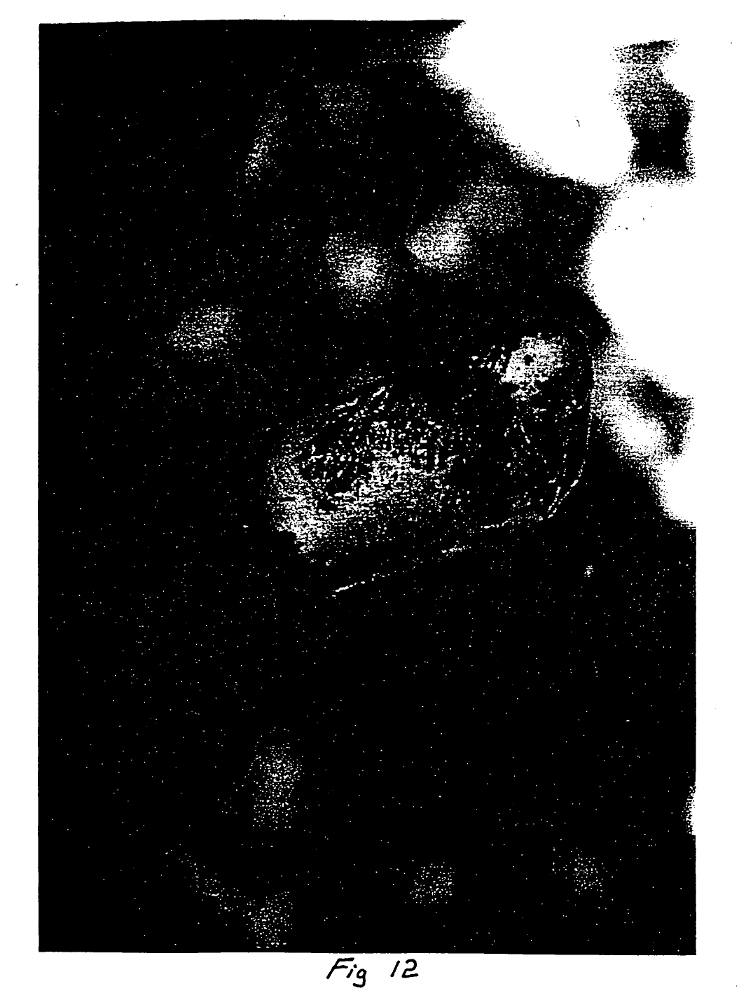

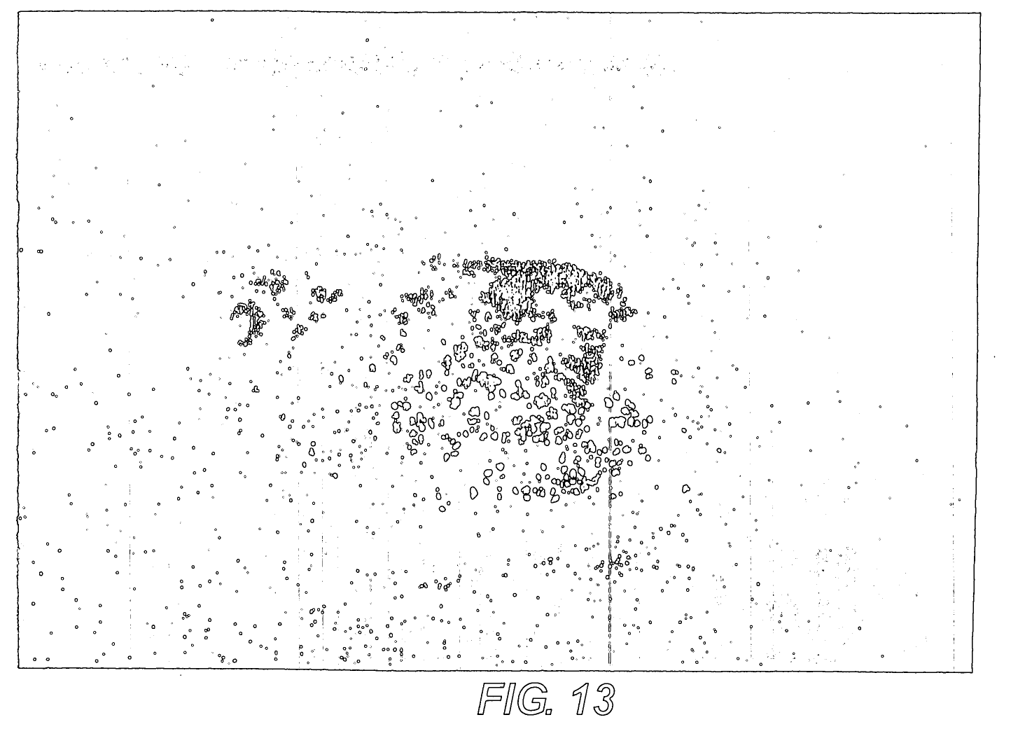

FIGS. 9 to 12 show a further example of the treatment of a horse with a penile tumor before, during and after treatment. FIG. 9 shows the horse prior to treatment. The horse was anesthetized and the tumor injected with the above formulation. FIG. 10 shows the response of the tumor to the composition. The tumor then separated entirely and fell off as shown in FIG. 11. FIG. 12 illustrates the penis after the treatment.

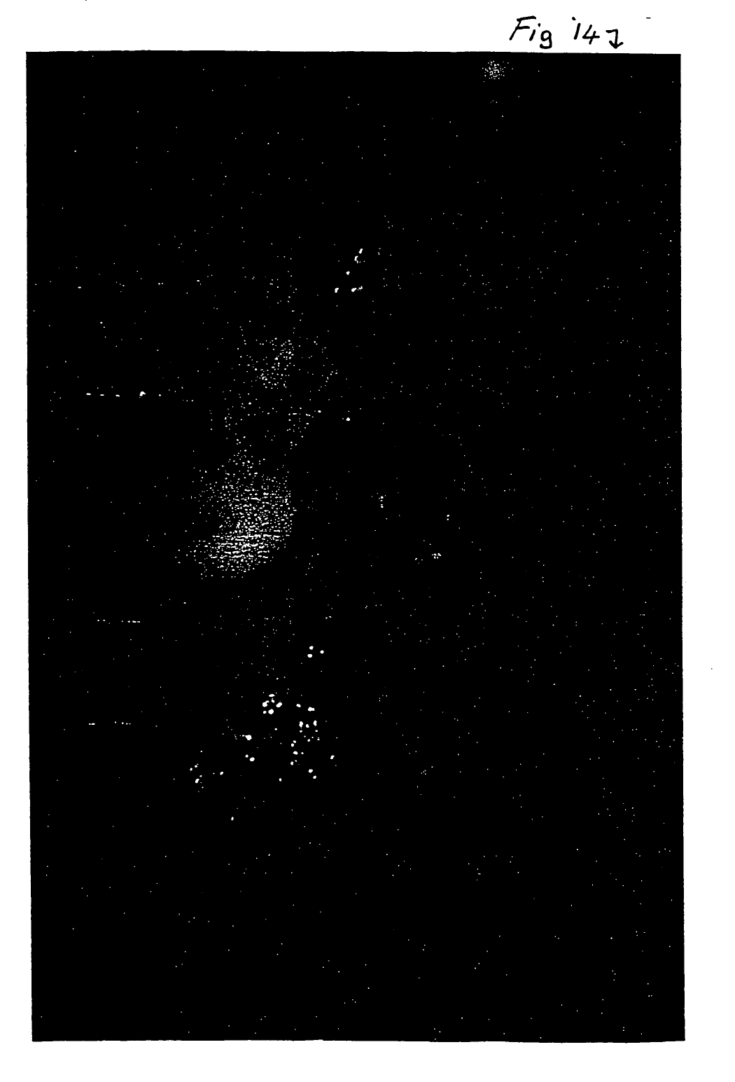

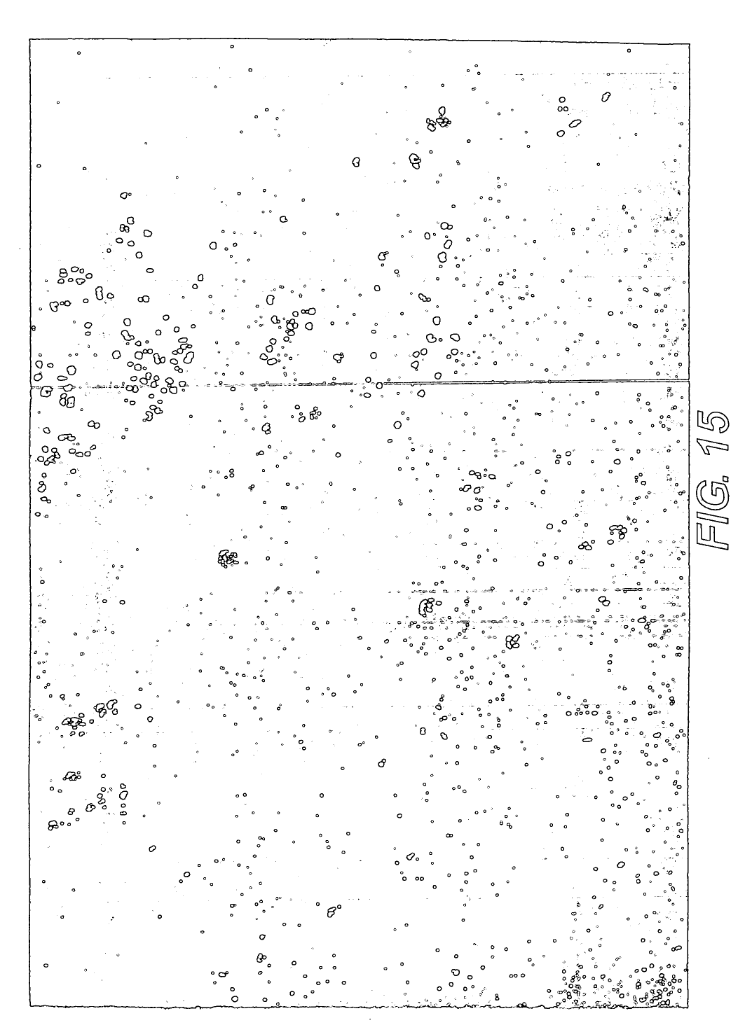

FIGS. 13, 14 and 15 show an example of the treatment of a human SCC. FIG. 13 shows the SCC located on the patient's scalp. The patient was treated with a single injection and recovery of the SCC shortly after treatment occurred as illustrated in FIGS. 14 and 15.

In the above examples, it can be seen that a composition of the present invention was successful in the treatment of solid sarcoids in animals and SCC in humans. During treatment, necrosis of the lesion was observed to begin almost immediately after injection.

It was also observed that similar treatment with BEC which contains free sugars was less effective than the inventive composition. Treatment with BEC required much higher dosages before any effect was observed.

The dosages of the compositions in the above examples is 100 mg of solasodine glycosides per 1 kg tumor. A typical tumor is 100 g such that a typical injection contains 10 mg solasodine glycosides. This dose for a 500 kg horse corresponds to 0.02 mg/kg body weight.

It can be seen that a therapeutic composition of the present invention provides a suprising and unexpected improvement in efficacy of glycoalkaloids in the treatment of cancers and tumors. This increase in efficacy allows disease conditions to be treated with dosages which are well below the threshold level of toxicity for normal cells. This is advantageous for the patient and also allows the inventive compositions to be used as diagnostic tools. Still further, the improved efficacy enables the duration of treatments to be reduced and total dosages to be decreased. This is advantageous for the overall safety and comfort of the patient and also provides a superior treatment regime in terms of cost effectiveness.

Throughout the specification (including claims if present) unless the context requires otherwise, the word “comprise” or variations such as “comprising” will be understood to imply the inclusion of a stated integer or group of integers but not the exclusion of any other integer or group of integers.

It will be appreciated that modifications and changes may be made to the embodiments described therein without departing from the spirit and scope of the invention as herein described.

What is claimed is:

1. A medicinal composition comprising at least one compound which can interact with a target cell, the at least one compound being a glycoalkaloid of the general formula I: 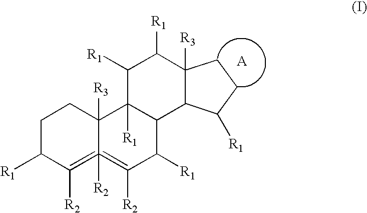
wherein:
either one of the dotted lines represents a double bond, and the other a single bond, or both represent single bonds;
A: represents a radical selected from the following radicals of general formulae (II) to (V): 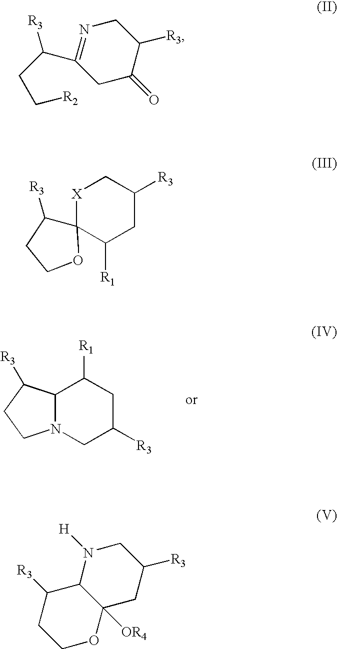
each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, alkyl and R⁴O-alkylene; each of R⁴ is a radical separately selected from the group consisting of hydrogen, carbohydrate and a carbohydrate derivative; “X” is a radical selected from the group comprising —CH₂—,—O— and —NH—;
wherein the compound includes at least one R⁴ group in which R⁴ is a carbohydrate or a derivative thereof;
together with a pharmaceutically acceptable carrier, adjuvant, excipient and/or diluent, wherein the composition is essentially free of sugars derived from the at least one glycoalkaloid.

2. The composition of claim 1, wherein R⁴ is selected from the group consisting of glyceric aldehyde; glycerose; erythrose; threose; ribose; arabinose; xylose; lyxose; altrose; allose; gulose; mannose; glucose; idose; galactose; talose; rhamnose; dihydroxyactone; erythrulose; ribulose; xylulose; psicose; fructose; sorbose; tagatose; and other hexoses (C₆H₁₂O₆); heptoses (C₇H₁₄O₇); octoses (C₈H₁₆O₈); nanoses (C₉H₁₈O₉); decoses (C₁₀H₂₀O₁₀); deoxysugars with branched chains (eg. apiose, hamamelose, streptose, cordycepose, mycarose and cladinose); compounds wherein the aldehyde, ketone or hydroxyl groups have been substituted (eg. N-acetyl, acetyl, methyl, replacement of CH₂OH); sugar alcohols; sugar acids; benzimidazoles; the enol salts of the carbohydrates; saccharinic acids; sugar phosphates.

3. The composition of claim 1, wherein the at least one glycoalkaloid is selected from the group consisting of solasonine, solamargine, and tomatine.

4. The composition of claim 1, wherein the at least one glycoalkaloid has been extracted from a plant source.

5. The composition of claim 4, wherein the plant source is from the Solanum genus.

6. The composition of claim 5, wherein the composition is a BEC mixture of solasodine glycosides.

7. The composition of claim 1, wherein the free sugar is rhamnose, or a disaccharide, trisaccharide, oligosaccharide or polysaccharide having rhamnose as a sugar moiety thereof.

8. The composition of claim 1 which is essentially free of any aglycone degradation product of the glycoalkaloid.

9. The composition of claim 1 in a form suitable for topical administration.

10. The composition of claim 9, which includes between about 0.001% to about 5 wt % of the at least one glycoalkaloid.

11. The composition of claim 1, which is in a form suitable for administration by injection.

12. The composition of claim 11, which includes a liquid carrier selected from the group consisting of DMSO, acetic acid and lactic acid.

13. The composition of claim 1, which includes a stablizing agent for stabilizing the at least one glycoalkaloid.

14. A method of preparing a glycoalkaloid preparation which comprises at least one glycoalkaloid of the general formula I: 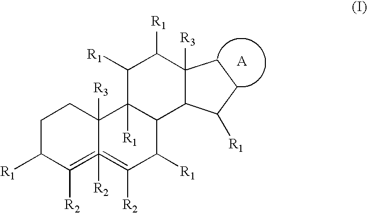
wherein:
either one of the dotted lines represents a double bond, and the other a single bond, or both represent single bonds;
A: represents a radical selected from the following radicals of general formulae (II) to (V): 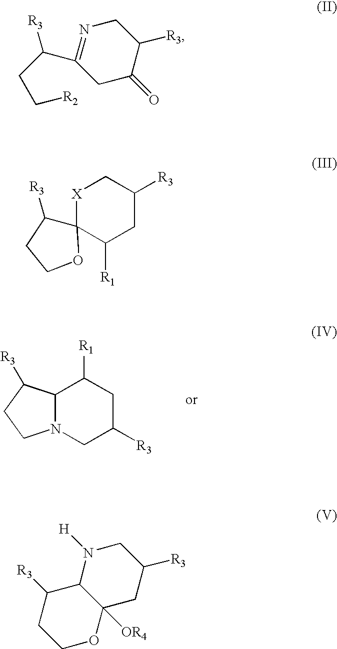
each of R¹ is a radical separately selected from the group consisting of hydrogen, amino, oxo and OR⁴; each of R² is a radical separately selected from the group consisting of hydrogen, amino and OR⁴; each of R³ is a radical separately selected from the group consisting of hydrogen, alkyl and R⁴O-alkylene; each of R⁴ is a radical separately selected from the group consisting of hydrogen, carbohydrate and a carbohydrate derivative;“X” is a radical selected from the group comprising —CH₂—,—O— and —NH—;
wherein the compound includes at least one R⁴ group in which R⁴ is a carbohydrate or a derivative thereof;
the method including extracting the at least one glycoalkaloid from a suitable plant material to form an extract and removing essentially all free sugars derived from the glycoalkaloid from the extract.

15. The method of claim 14, wherein the plant material is from the Solanum genus.

16. A method of preparing the composition of claim 1, including obtaining a glycoalkaloid preparation which comprises at least one glycoalkaloid according to general formula I and treating the preparation to remove essentially all of any free sugars from the preparation prior to addition of a pharmaceutically acceptable carrier, adjuvant, excipient and/or diluent.

17. The method of claim 15 wherein the preparation is further treated to remove any aglycone therefrom.

18. The method of claim 16, wherein the preparation is washed with an aqueous solvent.

19. The method of claim 16, wherein the glycoalkaloid preparation is extracted from a plant source.

20. The method of claim 18, wherein the plant source is from the Solanum genus.

21. The method of claim 18, wherein the glycoalkaloid preparation is a BEC mixture of solasodine glycosides.

22. The method of claim 18, wherein a time period of at least about 7 days has elapsed between the extraction and free sugar removal steps.

23. A method for the treatment or control of a condition selected from the group consisting of cancer, contraception, termination of pathogenic organisms and removal of abnormal cellular growth in a mammal requiring such treatment, the method comprising administering to said mammal an effective amount of the medicinal composition of claim 1.
